(19) United States
(12) Patent Application Publication
(10) Pub. No.: US 2026/0084168 A1
(43) Pub. Date: Mar. 26, 2026

(54) POWDER SPRAYER

(71) Applicant: CG Bio Co., Ltd., Seoul (KR)

(72) Inventors: Soon Won CHUNG, Yongin-si, Gyeonggido (KR); Jun Kyu PARK, Seongnam-si, Gyeonggi-do (KR); Min Soo KIM, Siheung-si, Gyeonggi-do (KR); Bo Mi MOON, Seongnam-si, Gyeonggido (KR)

(21) Appl. No.: 19/109,126

(22) PCT Filed: Feb. 24, 2022
(86) PCT No.: PCT/KR2022/002702

§ 371 Date: Mar. 5, 2025

(30) Foreign Application Priority Data
Apr. 26, 2021 (KR) 10-2021-0053690

(51) Int. Cl. B05B 7/14; A61M 31/00; B05B 7/24

(52) U.S. Cl. CPC B05B 7/1463; A61M 31/00; B05B 7/2491; A61M 2202/064

(57) ABSTRACT

The present application relates to a powder sprayer comprising: an air inlet part into which air is introduced from an external source; a powder mixing part to which powder is supplied from an external source; a confluence channel which is provided to join fluid moved from the powder mixing part and air moved from the air inlet part and discharge the joined air and fluid to the outside; a first flow channel which connects the air inlet part to the power mixing part; a second flow channel which connects the air inlet part to the confluence channel; a third flow channel which connects the powder mixing part to the confluence channel; and an on-off valve which is provided to selectively perform one of closing of the second flow channel and opening of at least one of the first flow channel and the second flow channel.

### TECHNICAL FIELD

The present disclosure relates to a powder sprayer.

### BACKGROUND ART

A powder sprayer is a device that supplies fluid containing air or powder into the catheter tube. Conventionally, there are various powder sprayers such as a powder sprayer that operates a pump by simply turning the power on/off to spray air pressure or spray powder, or a powder sprayer that sprays powder by vibrating the sprayer to improve this.

However, these sprayers have a disadvantage in that when a catheter is inserted into an endoscope channel and the sprayer is connected to the catheter to spray powder, the powder may fuse with the introduced liquid and block a catheter tube when liquid such as body fluid flows into the catheter tube through capillary action, which may cause a safety issue.

Japanese Patent Laid-Open No. 2020-191939 relates to a powder spraying device, and relates to a powder spraying device capable of discharging air or discharging powder and air.

### DISCLOSURE

### Technical Problem

The present disclosure is intended to solve the problems of the above-mentioned prior art, and to provide a powder sprayer capable of solving the problem of catheter blockage due to the mixing of powder and body fluid of the powder sprayer.

However, the technical tasks to be achieved by the exemplary embodiments of the present disclosure are not limited to the technical tasks described above, and other technical tasks may exist.

### Technical Solution

As a technical means for achieving the above technical task, according to a first aspect of the present disclosure, there is provided a powder sprayer including: an air inlet into which air is introduced from an outside; a powder mixing unit to which powder is supplied from the outside; a confluence channel provided to join fluid moved from the powder mixing unit and air moved from the air inlet and discharge the joined air and fluid to the outside; a first channel which connects the air inlet and the powder mixing unit; a second channel which connects the air inlet and the confluence channel; a third channel which connects the powder mixing unit and the confluence channel; and an on-off valve provided to selectively perform any one of closing the second channel and opening at least one of the first channel and the second channel.

According to one exemplary embodiment of the present disclosure, when the second channel is closed, powder may be supplied to the powder mixing unit and air may be supplied from the air inlet so that the powder and air are mixed and discharged through the confluence channel, but the present disclosure is not limited thereto.

According to one exemplary embodiment of the present disclosure, when at least one of the first channel and the second channel is open, the powder supply to the powder mixing unit may be stopped and air may be supplied from the air inlet so that the air may be discharged through the confluence channel, but the present disclosure is not limited thereto.

According to one exemplary embodiment of the present disclosure, the on-off valve may be a valve installed with respect to the second channel to selectively perform closing or opening of the second channel, but the present disclosure is not limited thereto.

According to one exemplary embodiment of the present disclosure, the air supplied to the powder mixing unit through the first channel may rise in a direction perpendicular to the powder mixing unit and then descend while rotating clockwise or counterclockwise, but the present disclosure is not limited thereto.

According to one exemplary embodiment of the present disclosure, the fluid moved from the powder mixing unit may include air and powder supplied to the powder mixing unit, but the present disclosure is not limited thereto.

According to one exemplary embodiment of the present disclosure, an angle between the powder mixing unit and the confluence channel may be 120° to 150°, but the present disclosure is not limited thereto.

According to one exemplary embodiment of the present disclosure, an angle between the first channel and the powder mixing unit may be 120° to 150°, but the present disclosure is not limited thereto.

According to one exemplary embodiment of the present disclosure, the powder sprayer may further include a check valve formed on the first channel and preventing air or body fluid introduced from the outside from flowing back toward the powder mixing unit, but the present disclosure is not limited thereto.

According to one exemplary embodiment of the present disclosure, the air inlet may include a pump for sucking the air and a filter, but the present disclosure is not limited thereto.

The above-described solving means are merely exemplary and should not be construed as limiting the present disclosure. In addition to the above-described exemplary embodiments, additional exemplary embodiments may exist in the drawings and detailed description of the disclosure.

### Advantageous Effects

Conventional powder sprayers spray air pressure or powder by operating a pump only by turning the power on/off, or through vibration. However, when a catheter is inserted into an endoscope channel and a sprayer is connected to the catheter to spray powder, a problem may occur where liquid such as body fluid in a catheter tube flows into the catheter or sprayer through capillary action, and the liquid fuses with the powder, thereby blocking the tube.

The powder sprayer according to the present disclosure is for non-vascular (digestive, or the like) applications, and even when powder is not sprayed, a small amount of air is discharged inside the catheter, and the discharge of the air can fundamentally suppress the capillary phenomenon in which the liquid flows into the catheter. Therefore, the powder sprayer according to the present disclosure can prevent the problem of the catheter tube being blocked by the mixing of body fluid and powder.

In addition, when excessive air is injected while using the powder sprayer according to the present disclosure, the air can be suctioned through the endoscope, thereby preventing danger.

In addition, in the powder sprayer according to the present disclosure, air is raised vertically and introduced into the powder mixing unit and then discharged downward in a spiral manner in a clockwise or counterclockwise direction, thereby improving the spraying speed of the powder by controlling the degree to which the powder is mixed into the air through a PCB mounted on the powder sprayer and the cyclone effect.

However, the effects that can be obtained from the present disclosure are not limited to the effects described above, and other effects may exist.

### DESCRIPTION OF DRAWINGS

FIG. 1 is a schematic diagram of a powder sprayer according to one exemplary embodiment of the present disclosure.

FIG. 2 is a schematic diagram of a powder sprayer according to one exemplary embodiment of the present disclosure.

FIG. 3 is a schematic diagram of a powder sprayer according to one exemplary embodiment of the present disclosure.

FIG. 4 is a schematic diagram of a powder mixing unit according to one exemplary embodiment of the present disclosure.

FIG. 5 is a schematic diagram of a powder mixing unit according to one exemplary embodiment of the present disclosure.

FIG. 6 is a graph illustrating performance of the powder sprayer according to an exemplary embodiment of the present disclosure and a conventional powder sprayer.

FIG. 7 is a graph illustrating the performance of the powder sprayer according to an exemplary embodiment of the present disclosure and the conventional powder sprayer.

FIG. 8 is a photograph of the conventional powder sprayer.

### BEST MODE

Hereinafter, exemplary embodiments of the present disclosure will be described in detail with reference to the attached drawings so that a person having ordinary knowledge in the technical field to which the present disclosure pertains can easily implement the present disclosure.

However, the present disclosure can be implemented in various different forms and is not limited to the exemplary embodiments described herein. In addition, in order to clearly describe the present disclosure in the drawings, parts that are not related to the description are omitted, and similar parts are given similar drawing reference numerals throughout the specification.

Throughout the present specification, when a part is said to be “connected” to another part, this includes not only the case where it is “directly connected” but also the case where it is “electrically connected” with another element in between.

Throughout the present specification, when it is said that an element is located “on,” “above,” “below,” “under,” or “below” another element, this includes not only cases where an element is in contact with another element, but also cases where another element exists between the two elements.

Throughout the present specification, whenever a part is said to “include” a component, this does not mean that it excludes other components, but rather that it may further include other components, unless otherwise specifically stated.

The terms “about,” “substantially,” and the like used in this specification are used in a meaning that is at or close to the numerical value when manufacturing and material tolerances inherent in the meanings mentioned are presented, and are used to prevent unscrupulous infringers from unfairly utilizing the disclosure in which exact or absolute values are mentioned to help understanding the present disclosure. In addition, throughout the present specification, “a step of doing ˜” or “a step of ˜” does not mean a “step for ˜”.

Throughout the present specification, the term “combination of these” included in the expressions in the Markush format means a mixture or combination of one or more selected from the group consisting of the components described in the Markush format, and means including one or more selected from the group consisting of said components.

Throughout the present specification, references to “A and/or B” mean “A or B, or A and B”.

Hereinafter, a powder sprayer of the present disclosure will be specifically described with reference to implementation embodiments, exemplary embodiments, and drawings. However, the present disclosure is not limited to these implementation embodiments, exemplary embodiments, and drawings.

As a technical means for achieving the above-mentioned technical task, a first aspect of the present disclosure relates to a powder sprayer including an air inlet 100 into which air is introduced from an outside, a powder mixing unit 200 to which powder is supplied from the outside, a confluence channel 440 provided to join fluid moved from the powder mixing unit 200 and air moved from the air inlet 100 and discharge the joined air and fluid to the outside, a first channel 410 which connects the air inlet 100 and the powder mixing unit 200, a second channel 420 which connects the air inlet 100 and the confluence channel 440, a third channel which connects the powder mixing unit 200 and the confluence channel 440, and an on-off valve 300 provided to selectively perform any one of closing the second channel 420 and opening at least one of the first channel 410 and the second channel 420.

According to one exemplary embodiment of the present disclosure, the fluid moved from the powder mixing unit 200 may include air and powder supplied to the powder mixing unit 200, but the present disclosure is not limited thereto.

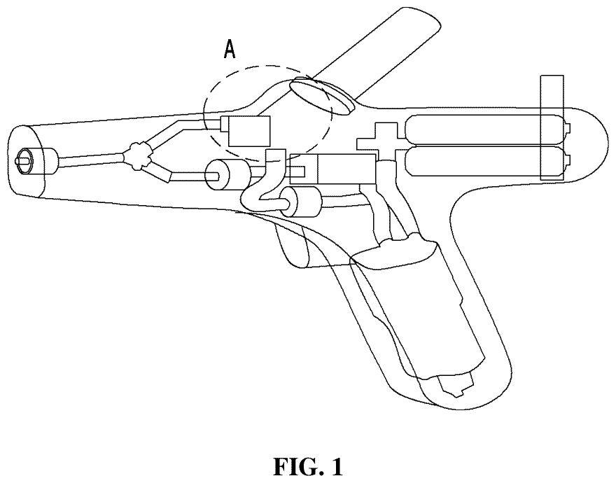

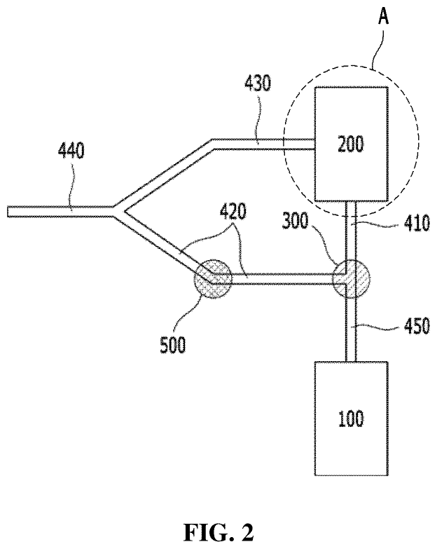

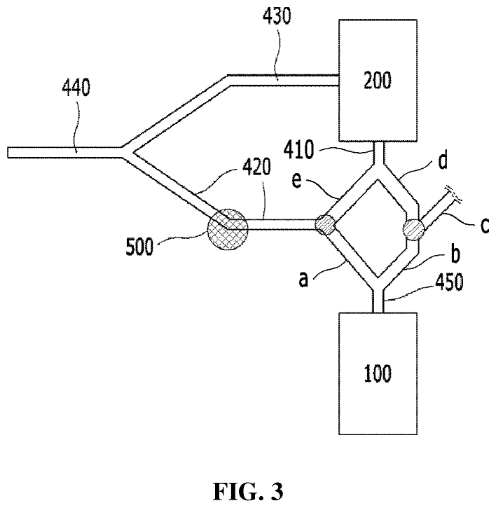

FIGS. 1 to 3 are schematic diagrams of a powder sprayer according to one exemplary embodiment of the present disclosure, in which FIG. 1 is a 3D drawing of the powder sprayer, and FIGS. 2 and 3 only represent partial schematic diagrams of the powder sprayer. As will be described later, FIG. 2 is a schematic diagram of a powder sprayer having a structure in which an air channel 450 is extended from the air inlet 100 and then separated into the first channel 410 and the second channel 420 by the on-off valve 300, and FIG. 3 is a schematic diagram of a powder sprayer having a structure in which the air channel 450 is separated into two channels a and b, and then the on-off valve 300 is formed in each of the two channels a and b.

According to one exemplary embodiment of the present disclosure, the powder sprayer 10 may be connected to a catheter, but the present disclosure is not limited thereto. Specifically, the catheter may be connected to the confluence channel 440.

When a catheter is inserted into an endoscope channel and the catheter is connected to a general powder sprayer to supply powder, a problem occurs in which liquid such as body fluid within a catheter tube flows into the catheter through capillary action. In this case, when the liquid and the powder are mixed, the powder and liquid may harden or coagulate, causing a problem in which the catheter is blocked.

In order to solve the above problem, the powder sprayer according to the present disclosure may prevent the phenomenon of the liquid flowing into the catheter through the capillary phenomenon by continuously releasing a small amount of air even when the powder is not sprayed through the catheter tube.

Specifically, air is introduced into the air inlet 100 from the outside of the powder sprayer. The introduced air may be discharged to the outside via the first channel 410 and/or the second channel 420. The air that has entered into the first channel 410 moves to the powder mixing unit 200, and at this time, the powder introduced from the outside of the powder mixing unit 200 or the powder contained in the powder mixing unit 200 and the air may be mixed and discharged to the confluence channel 440 via the third channel 430. Meanwhile, the air that has entered into the second channel 420 is discharged to the confluence channel 440. That is, the air that has entered into the confluence channel 440 includes a fluid in which the powder and air are mixed via the powder mixing unit 200 and/or air introduced from the air inlet 100.

The above-described on-off valve 300 is for controlling the opening/closing degree of the first channel 410 and the second channel 420, and may be a solenoid valve. For example, the on-off valve 300 may control the opening/closing degree of the first channel 410 in a state where the second channel 420 is always open, control the opening/closing degree of the second channel 420 in a state where the first channel 410 is always open, or control the opening/closing degrees of the first channel 410 and the second channel 420, thereby controlling the degree to which the introduced air passes through the first channel 410 and the degree to which the introduced air passes through the second channel 420, but the present disclosure is not limited thereto. For example, the on-off valve 300 is formed on the air channel 450 before the first channel 410 and the second channel 420 are branched off as illustrated in FIG. 2, thereby controlling the amount of air flowing in the first channel 410 and the second channel 420.

For another example, as illustrated in FIG. 3, the on-off valves 300 may be formed in each of a channel b and the second channel 420. In this case, a first on-off valve 310 formed on the channel b performs the function of passing air introduced from the air channel 450 to the powder mixing unit 200 or discharging the air to the outside of the powder sprayer 10, and a second on-off valve 300 formed on the second channel 420 may pass air introduced from the air channel 450 to the second channel 420, to the confluence channel 440, or transport the air to the powder mixing unit 200 through a channel (channel e) formed between the second channel 420 and the powder mixing unit 200.

In the case of the powder sprayer of FIG. 3, when power is applied to the first on-off valve 310 formed between the channel b and a channel d toward the first channel 410 from the air inlet 100, air may be supplied to the powder mixing unit 200 through the first channel 410, and the fluid in which the powder and air are mixed in the powder mixing unit 200 may be discharged to the outside through the third channel 430. In this case, all of the air pressure generated in the air inlet 100 may be transferred to the powder mixing unit 200. In addition, by controlling the power applied to the first on-off valve 310, the entire air from the air inlet 100 toward the powder mixing unit 200 is discharged to the outside of the powder sprayer, that is, through the channel c, without passing through the channel d, thereby controlling the amount of air introduced into the powder mixing unit 200, thereby controlling the amount of powder sprayed.

In addition, even when the voltage applied to the first on-off valve 310 is completely blocked, by controlling the voltage applied to the second on-off valve 320, it is possible to control whether the fluid discharged from the confluence channel 440 is air (when the entire air passing through the channel a passes through the second channel 420) or the mixture of air and powder (when at least a portion of the air passing through the channel a passes through channel e).

Although not indicated by a separate symbol in FIGS. 2 and 3, a point where the second channel 420 and the third channel 430 join to form the confluence channel 440, a point where the air channel 450 branches into the channel a and the channel b, and the point where the channel e and the channel d join are Y connectors.

According to one exemplary embodiment of the present disclosure, when at least one of the first channel 410 and the second channel 420 is open, the powder supply to the powder mixing unit 200 is stopped and air is provided from the air inlet 100, so that the air may be discharged through the confluence channel 440, but the present disclosure is not limited thereto.

According to one exemplary embodiment of the present disclosure, when at least one of the first channel 410 and the second channel 420 is open, the powder of the powder mixing unit 200 and the air provided from the air inlet 100 are mixed to form the fluid, or air is provided from the air inlet 100, so that the fluid or the air provided from the air inlet 100 may be discharged through the confluence channel 440, but the present disclosure is not limited thereto.

For example, when the first channel 410 is closed and the second channel 420 is open, the entire air introduced into the air inlet 100 may be transferred to the confluence channel 440 via the second channel 420, regardless of whether the powder in the powder mixing unit 200 is supplied externally or already placed internally.

For example, when the first channel 410 is open and the second channel 420 is closed, the fluid supplied to the catheter may vary depending on whether the powder of the powder mixing unit 200 is supplied externally or is already placed inside. In a case where the powder of the powder mixing unit 200 is supplied from the outside of the powder sprayer, when powder is not supplied to the powder mixing unit 200 from the outside of the powder sprayer, the fluid passing through the third channel 430 is air, and when powder is supplied to the powder mixing unit 200 from the outside, the fluid passing through the third channel 430 may include air and powder. Meanwhile, when powder is placed inside the powder mixing unit 200, the air introduced through the first channel 410 and the powder inside the powder mixing unit 200 are mixed, so that the fluid passing through the third channel 430 may include air and powder. That is, depending on whether the powder mixing unit 200 contains powder or whether powder is supplied to the powder mixing unit 200, the fluid supplied to the confluence channel 440 may contain air and optionally contain the powder.

In another example, when both the first channel 410 and the second channel 420 have a partially open structure, the air introduced into the air inlet 100 may be distributed into air transferred to the powder mixing unit 200 via the first channel 410 and air transferred to the second channel 420 depending on the degree of opening of the first channel 410 and the second channel 420. In this case, the air passing through the first channel 410 may or may not be mixed with the powder in the powder mixing unit 200, and the air passing through the second channel 420 is transferred to the confluence channel 440, and the fluid or air including the powder and air passing through the powder mixing unit 200 and the air passing through the second channel 420 may be mixed in the confluence channel 440 and discharged to the outside.

According to one exemplary embodiment of the present disclosure, the on-off valve 300 may be provided to be installed with respect to the second channel 420 to selectively perform closing or opening of the second channel 420, but the present disclosure is not limited thereto.

According to one exemplary embodiment of the present disclosure, the on-off valve 300 may be provided to be installed with respect to the first channel 410 to selectively perform closing or opening of the first channel 410, but the present disclosure is not limited thereto.

According to one exemplary embodiment of the present disclosure, the on-off valve 300 is installed at a point where the first channel 410 and the second channel 420 branch off, and may control the opening of the first channel 410 and the second channel 420, but the present disclosure is not limited thereto. In this case, when the on-off valve 300 is installed at the branching point, the degree to which the first channel 410 is open is the same as the degree to which the second channel 420 is closed, and the degree to which the first channel 410 is closed is the same as the degree to which the second channel 420 is open.

For example, the on-off valve 300 may be formed at the point where the first channel 410 and the second channel 420 branch from the air channel 450 extending from the air inlet 100, as illustrated in FIG. 2, or may include the first on-off valve 310 and the second on-off valve 320 formed on the channel a and the channel b, respectively, branched from the air channel 450, as illustrated in FIG. 3.

However, when the on-off valve 300 is formed only on the first channel 410, it may be difficult to control the amount of air passing through the first channel 410 because the air always tries to escape toward the second channel 420.

According to one exemplary embodiment of the present disclosure, the air supplied to the powder mixing unit 200 through the first channel 410 may rise in a direction perpendicular to the powder mixing unit 200 and then descend while rotating clockwise or counterclockwise, but the present disclosure is not limited thereto.

According to one exemplary embodiment of the present disclosure, the powder mixed with air in the powder mixing unit 200 may include powder supplied from the outside during the operation of the powder sprayer, or powder stored inside the powder mixing unit 200, but the present disclosure is not limited thereto. When the powder mixed with air in the powder mixing unit 200 includes powder stored inside the powder mixing unit 200, the powder mixing unit 200 may include a powder control unit (not shown) that stores and manages the powder stored inside.

The powder mixing unit 200 may include a vertical rising channel (not shown) extending from the first channel 410 and allowing air to rise in a direction perpendicular to the powder mixing unit 200 and a rotary discharge channel (not shown) allowing the air to descend while rotating clockwise or counterclockwise.

In general, when a fluid passes through a narrow gap, the density of the fluid increases or velocity thereof increases, which may lower the pressure and cause a Venturi effect, which sucks substances within the passage through which the fluid passes. In addition, when the fluid proceeds while making a swirling motion, a cyclone capture effect, which causes particles within the fluid to escape the swirling flow and be collected by the centrifugal force generated by the swirling motion, may occur.

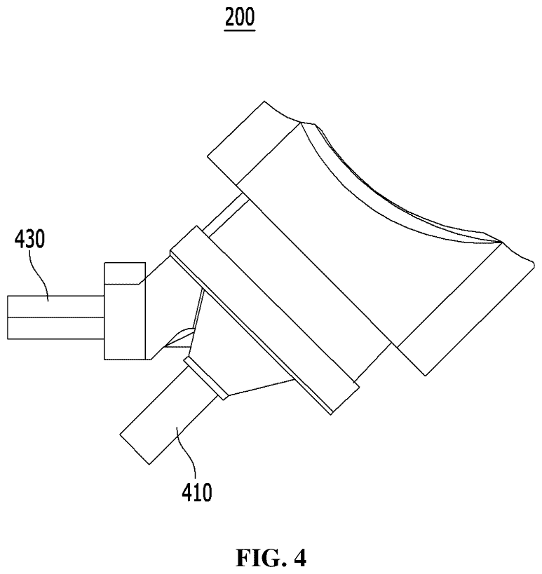

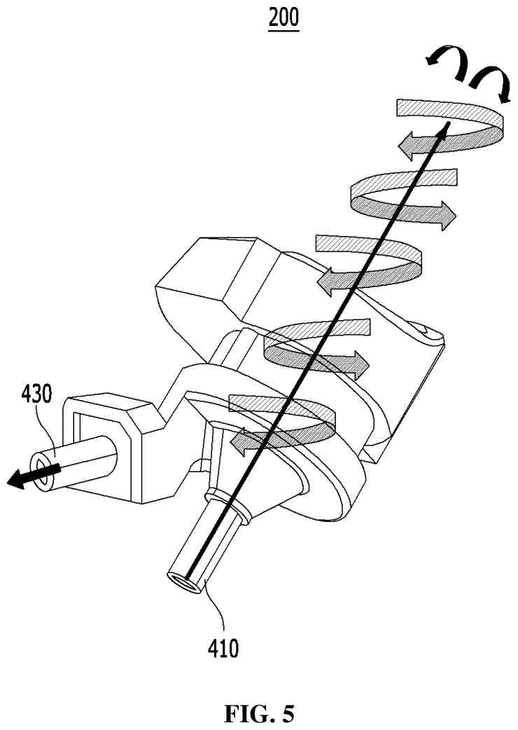

FIGS. 4 and 5 are schematic diagrams of the powder mixing unit according to one exemplary embodiment of the present disclosure. In this regard, the powder mixing unit 200 of FIGS. 4 and 5 corresponds to a part A of FIGS. 1 and 2, and referring to FIGS. 1, 4, and 5, the powder mixing unit is coupled in an inclined state with respect to the powder sprayer. In this regard, although the part A is not separately indicated in FIG. 3, the relationship between the first channel 410, the powder mixing unit 200, and the third channel 430 of FIG. 3 is also the same as in FIGS. 1, 2, 4, and 5.

In this case, the air introduced into the powder mixing unit rises vertically and then descends while rotating in a spiral direction and is discharged together with the powder.

Specifically, the air that rises vertically within the powder mixing unit 200 through the first channel 410 collides with the wall of a glass tube (Vial), descends while rotating counterclockwise, and is discharged along the third channel 430 that extends counterclockwise along the side surface of the first channel 410. At this time, powder may be mixed together with the discharge of the air. That is, the fluid flowing through the third channel 430 may include air and powder.

In the powder sprayer according to the present disclosure, the air and powder may be mixed in the process in which pure air introduced from the air inlet 100 rises through the vertical rising channel and then rotates along the rotary discharge channel. Specifically, before or after the air enters the rotary discharge channel for the rotational motion, powder is introduced into the rotary discharge channel from the outside of the powder sprayer or the powder is discharged into the rotary discharge channel by the powder control unit, and thus, the air and powder introduced into the powder mixing portion 200 may be mixed through the generated cyclone effect.

According to one exemplary embodiment of the present disclosure, an angle between the powder mixing unit 200 and the confluence channel 440 may be 120° to 150°, but the present disclosure is not limited thereto.

According to one exemplary embodiment of the present disclosure, an angle between the first channel 410 and the powder mixing unit 200 may be 120° to 150°, but the present disclosure is not limited thereto.

Preferably, the angle between the powder mixing unit 200 and the confluence channel 440 may be 135°, and the angle between the first channel 410 and the powder mixing unit 200 may be 135°, but the present disclosure is not limited thereto.

The angle between the first channel 410 and the powder mixing unit 200 may mean the angle between the first channel 410 and the vertical rising channel, and the angle between the powder mixing unit 200 and the confluence channel 440 is the same as the angle between the vertical rising channel and the third channel 430.

In this regard, in a conventional powder sprayer, the angle between the powder mixing unit and the channel extending from the powder mixing unit to the external catheter is vertical, but in the powder sprayer 10 according to the present disclosure, the angle between the powder mixing unit 200 and the third channel 430 extending from the powder mixing unit 200 is 135°, and air in the first channel 410 may be vertically introduced into the powder mixing unit 200, which can be confirmed through FIGS. 4 and 5, which specifically illustrates the part A of FIGS. 1 and 2.

According to one exemplary embodiment of the present disclosure, a check valve 500 formed on the second channel 420 and preventing a fluid containing powder and air supplied from the powder mixing unit 200 from flowing back into the second channel 420 may be additionally included, but the present disclosure is not limited thereto. Preferably, the check valve 500 may be formed between a point where the second channel 420 and the third channel 430 meet and the confluence channel 440 begins, and a point where the second channel 420 begins from the air inlet 100. More preferably, the check valve 500 may be formed at a point where the second channel 420 is bent, as illustrated in FIG. 2.

In this regard, since the check valve 500 is formed on the second channel 420, it is possible to suppress the phenomenon in which the fluid moved from the powder mixing unit 200 is not discharged to the confluence channel 440 but flows back to the second channel 420, and control whether the fluid is discharged to the outside.

According to one exemplary embodiment of the present disclosure, the air inlet 100 may include a pump for sucking the air and a filter, but the present disclosure is not limited thereto.

The present disclosure will be described in more detail through the following experimental examples, but the following exemplary embodiments are for the purpose of explanation only and are not intended to limit the scope of the present disclosure.

### Experimental Embodiment

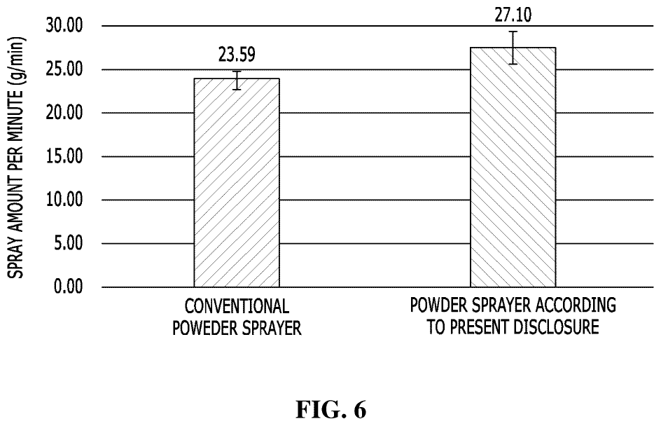

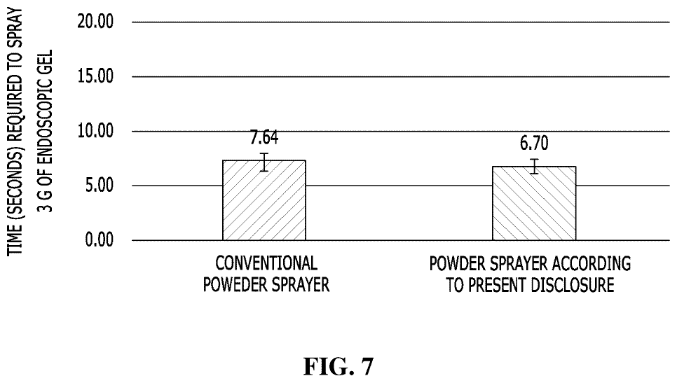

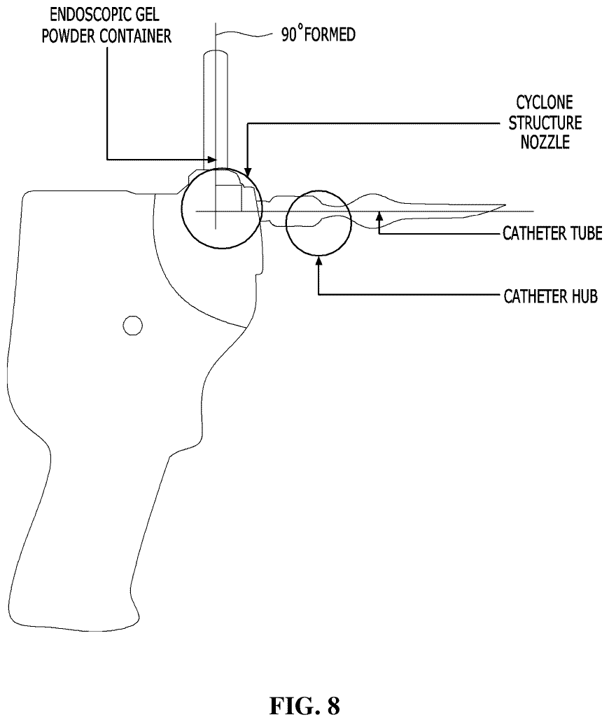

FIGS. 6 and 7 are graphs illustrating the performance of the powder sprayer according to an exemplary embodiment of the present disclosure and the conventional powder sprayer, and FIG. 8 is a photograph of the conventional powder sprayer.

In the conventional powder sprayer (vertical type) in which the angle between the powder mixing unit and the third channel extending from the powder mixing unit and supplying powder and air to the catheter is vertical, and the powder sprayer according to the present disclosure in which the angle between the powder mixing unit and the third channel extending from the powder mixing unit is 135°, a powder spray amount per minute and a time required to spray 3 g of endoscopic gel powder were compared.

TABLE 1

| | | | | Average | |
|---|---|---|---|---|---|
| | | | | spray | |
| | Spray amount per | | | amount | |
| | minute (g/min) | | | (g/min) | Standard |
| Classification | 1 time | 2 times | 3 times | Avg. | deviation |
| Conventional | 23.77807 | 22.38806 | 24.590164 | 23.59 | 1.113619 |
| powder sprayer | | | | | |
| (Vertical type) | | | | | |
| Powder sprayer | 29.03226 | 26.86567 | 25.416667 | 27.10 | 1.819625 |
| according to | | | | | |
| present | | | | | |
| disclosure | | | | | |

TABLE 2

| | | | | Average time | |
|---|---|---|---|---|---|
| | Time (seconds) taken to | | | (seconds) | |
| | spray 3 g of powder | | | taken | Standard |
| Classification | 1 time | 2 times | 3 times | Avg. | deviation |
| Conventional | 7.57 | 8.04 | 7.32 | 7.64 | 0.37 |
| powder sprayer | | | | | |
| (Vertical type) | | | | | |
| Powder sprayer | 6.2 | 6.7 | 7.2 | 6.70 | 0.50 |
| according to | | | | | |
| present | | | | | |
| disclosure | | | | | |

Referring to FIGS. 6 to 8 and Tables 1 and 2, it can be confirmed that when the angle between the first and third channels is 135° (powder sprayer according to the present disclosure), powder may be sprayed at a faster speed than a powder sprayer (conventional powder sprayer) in which the angle between the first and third channels is 90°.

The above description of the present disclosure is for illustrative purposes only, and those skilled in the art will understand that the present disclosure can be easily modified into other specific forms without changing the technical idea or essential features of the present disclosure. Therefore, it should be understood that the exemplary embodiments described above are exemplary in all respects and not restrictive. For example, each component described as a single component may be implemented in a distributed manner, and likewise, components described as distributed may be implemented in a combined manner.

The scope of the present application is indicated by the patent claims described below rather than the detailed description above, and all changes or modifications derived from the meaning and scope of the patent claims and their equivalent concepts should be interpreted as being included in the scope of the present application.

1. A powder sprayer comprising:
an air inlet into which air is introduced from an outside;
a powder mixing unit to which powder is supplied from the outside;
a confluence channel provided to join fluid moved from the powder mixing unit and air moved from the air inlet and discharge the joined air and fluid to the outside;
a first channel which connects the air inlet and the powder mixing unit;
a second channel which connects the air inlet and the confluence channel;
a third channel which connects the powder mixing unit and the confluence channel; and
an on-off valve provided to selectively perform any one of closing the second channel and opening at least one of the first channel and the second channel.

2. The powder sprayer of claim 1, wherein when the second channel is closed, the powder is supplied to the powder mixing unit and the air is supplied from the air inlet so that the powder and air are mixed and discharged through the confluence channel, and
when at least one of the first channel and the second channel is open, the powder supply to the powder mixing unit is stopped and the air is supplied from the air inlet so that the air is discharged through the confluence channel.

3. The powder sprayer of claim 2, wherein the on-off valve is a valve installed with respect to the second channel to selectively perform the closing or opening of the second channel.

4. The powder sprayer of claim 1, wherein the air supplied to the powder mixing unit through the first channel rises in a direction perpendicular to the powder mixing unit and then descends while rotating clockwise or counterclockwise.

5. The powder sprayer of claim 4, wherein the fluid moved from the powder mixing unit includes air and powder supplied to the powder mixing unit.

6. The powder sprayer of claim 1, wherein an angle between the powder mixing unit and the confluence channel is 120° to 150°.

7. The powder sprayer of claim 1, wherein an angle between the first channel and the powder mixing unit is 120° to 150°.

8. The powder sprayer of claim 1, further comprising a check valve formed on the first channel and preventing the fluid moved from the powder mixing unit from flowing back toward the first channel.

9. The powder sprayer of claim 1, wherein the air inlet includes a pump for sucking the air and a filter.
